(19) United States
(12) Patent Application Publication
(10) Pub. No.: US 2008/0312450 A1
(43) Pub. Date: Dec. 18, 2008

(54) Method for Start-Up of Oxidation Catalysts

(75) Inventors: Samuel Neto, Dresden (DE); Frank Rosowski, Mannheim (DE); Sebastian Storck, Mannheim (DE); Juergen Zuehlke, Speyer (DE); Hans-Martin Allmann, Neunkirchen (DE); Thomas Lautensack, Birkenau (DE); Rainer Steeg, Goennheim (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 11/994,516

(22) PCT Filed: Jun. 30, 2006
(86) PCT No.: PCT/EP2006/064762

§ 371 Date: Mar. 12, 2008

(30) Foreign Application Priority Data
Jul. 4, 2005 (DE) 10 2005 031 465.1

(51) Int. Cl. C07D 213/79; C07C 27/10; C07D 307/60; C07D 307/89; C07C 51/16

(52) U.S. Cl. 546/318; 562/512.2; 549/258; 549/248; 562/480; 562/512.4

(57) ABSTRACT

Methods comprising: providing an oxidation catalyst bed; and starting up the oxidation catalyst at a temperature of 360° C. to 400° C. with an amount of air of 1.0 to 3.5 standard m³/h and a hydrocarbon loading of 20 to 65 g/standard m³, such that a hot spot having a temperature of 390° C. to <450° C. is formed in the first 7-20% of the catalyst bed.

The present invention relates to a method of starting up oxidation catalysts, which comprises starting up the catalysts at a temperature of from 360° C. to 400° C. using an amount of air of from 1.0 to 3.5 standard m³/h and a hydrocarbon loading of from 20 to 65 g/standard m³, resulting in formation of a hot spot having a temperature of from 390° C. to <450° C. in the first 7-20% of the catalyst bed.

Many aldehydes, carboxylic acids and/or carboxylic anhydrides are prepared industrially by catalytic gas phase oxidation of aromatic hydrocarbons such as benzene, o-, m- or p-xylene, naphthalene, toluene or durene (1,2,4,5-tetra-methylbenzene) in fixed-bed reactors, preferably shell-and-tube reactors. Depending on the starting material, the product obtained is, for example, benzaldehyde, benzoic acid, maleic anhydride, phthalic anhydride, isophthalic acid, terephthalic acid or pyromellitic anhydride. Catalysts based on vanadium oxide and titanium dioxide are predominantly used for this purpose.

The gas-phase oxidation is strongly exothermic. Local temperature maxima, known as hot spots, in which a higher temperature than in the remainder of the catalyst bed prevails are formed. Above a certain hot spot temperature, the catalyst can be damaged irreversibly.

All catalysts lose activity as time goes on as a result of aging processes. This makes itself particularly apparent in the main reaction zone, i.e. in the first catalyst zone nearest the gas inlet, since the highest thermal stress occurs there. During the life of the catalyst the main reaction zone moves ever deeper into the catalyst bed. This results in intermediates and by-products no longer being able to be reacted completely since the main reaction zone is now also located in catalyst zones which are less selective and more active. The product quality of the phthalic anhydride produced thus deteriorates to an increasing extent. The slowing of the reaction and thus the deterioration in the product quality can be countered by increasing the reaction temperature, for example by increasing the salt bath temperature, and/or by increasing the amount of air. However, this temperature increase is associated with a decrease in the yield of phthalic anhydride.

The position and temperature of the hot spots can be controlled, for example, by the start-up of the oxidation catalysts.

DE-A 22 12 947 describes a process for preparing phthalic anhydride in which the salt bath is set to a temperature of from 373 to 410° C. at the beginning, at least 1000 liters per hour of air and at least 33 g of o-xylene per standard m³ of air are passed through a tube so that a hot spot temperature of from 450 to 465° C. is established in the first third of the catalyst bed, calculated from the point at which the gas enters.

DE-A 25 46 268 discloses a process for preparing phthalic anhydride, in which the process is carried out at a salt bath temperature of from 360 to 400° C. and an amount of air of 4.5 standard m³ at a loading of from 36.8 to 60.3 g of o-xylene per standard m³.

DE-A 198 24 532 describes a process for preparing phthalic anhydride, in which the o-xylene loading is increased from 40 to 80 g per standard m³ over a running-up time of a number of days at an amount of air of 4.0 standard m³.

EP-B 985 648 discloses a process in which phthalic anhydride is prepared at an amount of air of from 2 to 3 standard m³ and an o-xylene loading of from 100 to 140 g per standard m³.

Despite the results achieved in the setting of the position and temperature of the hot spot, there continues to be a need for optimization because of the great importance of these two factors in the deactivation of catalysts.

It is therefore an object of the invention to discover a method of starting up oxidation catalysts which further slows the deactivation of the catalysts.

We have accordingly found a method of starting up oxidation catalysts, which comprises starting up the catalysts at a temperature of from 360° C. to 400° C. using an amount of air of from 1.0 to 3.5 standard m³/h and a hydrocarbon loading of from 20 to 65 g/standard m³, resulting in formation of a hot spot having a temperature of from 390° C. to <450° C. in the first 7-20% of the catalyst bed.

The oxidation catalysts are advantageously started up at an amount of air of from 1.5 to <4.0 standard m³/h, preferably from 1.5 to 3.5 standard m³/h, particularly preferably from 2.5 to 3.5, in particular at an amount of air of from 3.0 to 3.5 standard m³/h.

The amount of air is advantageously increased slowly during start-up. The increase in the amount of air advantageously takes place after from 2 to 48 hours, preferably from 10 to 26 hours. The increase in the amount of air is advantageously carried out in steps of 0.05-0.5 standard m³/h. The increase in the amount of air is generally carried out either in equidistant steps or firstly in relatively small steps and then, as the amount of air increases, in larger steps. During the increase in the amount of air, phases during which the amount of air introduced is constant can be present. The amount of air during operation, or the target amount of air, is advantageously 4.0 standard m³/h.

The hydrocarbon loading is advantageously from 25 to 60 g/standard m³, preferably from 30 to 55 g/standard m³, in particular from 30 to 45 g/standard m³.

The hydrocarbon loading is advantageously increased slowly during start-up. Basically, the loading can be increased when a stable hot spot temperature profile has been established. The increase in the hydrocarbon loading advantageously takes place after a start-up time of from 5 to 60 minutes. The increase in the hydrocarbon loading is advantageously carried out in steps of 0.5-10 g/standard m³. The increase in the loading is advantageously carried out firstly in relatively large steps and then, at a higher loading, in smaller steps. During the increase in the hydrocarbon loading, phases during which the hydrocarbon loading is constant can be present. The hydrocarbon loading during operation, or the target-carbon loading, is advantageously from 70 to 120 g/standard m³.

The increase in the amount of air can be effected synchronously or asynchronously to the increase in the hydrocarbon loading. When the increase in the amount of air is carried out asynchronously with the increase in the loading, it is advantageous to increase the loading first and then to increase the amount of air.

Start-up is advantageously carried out so that the hot spot is formed in the first zone comprising the first 10-20% of the total catalyst bed. For example, the hot spot is formed in the first 30-60 cm at a total catalyst bed of 300 cm. The hot spot is preferably formed in the first 13-20% of the total catalyst bed.

The catalyst bed advantageously consists of a plurality of zones composed of catalysts having differing activities and selectivities, with the catalyst activity advantageously increasing from the gas inlet to the gas outlet. If appropriate, one or more catalyst zones which are located upstream or in between and have a higher activity than the next zone in the direction of gas flow can be used. Use is customarily made of from two to six catalyst zones, in particular from three to five.

The first zone advantageously makes up from 30 to 60 percent of the total catalyst bed. The fewer zones a catalyst system has, the larger the first zone as a proportion of the total catalyst bed.

The hot spot temperature in the first zone is advantageously from 420 to <450° C. after 24 hours.

The start-up of the oxidation catalysts is usually carried out at a gauge pressure of from 0 to 0.45 barg at the inlet.

In a preferred embodiment of a multizone layered catalyst system for preparing phthalic anhydride, the first zone nearest the gas inlet, i.e. the least active zone, comprises a catalyst on a nonporous and/or porous support material having from 7 to 11% by weight, based on the total catalyst, of active composition comprising from 4 to 11% by weight of V₂O ₅, from 0 to 4% by weight of Sb₂O₃ or Nb₂O₅, from 0% by weight to 0.3% by weight of P, from 0.1 to 1.1% by weight of alkali (calculated as alkali metal) and TiO₂ in anatase form as balance, with preference being given to using cesium as alkali metal.

The titanium dioxide in anatase form which is used advantageously has a BET surface area of from 5 to 50 m²/g, in particular from 15 to 30 m²/g. It is also possible to use mixtures of titanium dioxide in anatase form having different BET surface areas, with the proviso that the resulting BET surface area is from 15 to 30 m²/g. The individual catalyst zones can also comprise titanium dioxide having different BET surface areas. The BET surface area of the titanium dioxide used preferably increases from the first zone nearest the gas inlet to the last zone nearest the gas outlet.

Support materials used are advantageously spherical, ring-shaped or shell-shaped supports comprising a silicate, silicon carbide, porcelain, aluminum oxide, magnesium oxide, tin dioxide, rutile, aluminum silicate, magnesium silicate (steatite), zirconium silicate or cerium silicate or mixtures thereof. Coated catalysts in which the catalytically active composition is applied in the form of a shell to the support have been found to be particularly useful.

The compositions of the further catalyst zones for preparing phthalic anhydride are known to those skilled in the art and are described, for example, in WO 04/103944.

The invention further provides oxidation catalysts which are produced by the method of the invention. For example, the invention provides oxidation catalysts for preparing carboxylic acids and/or carboxylic anhydrides by catalytic gas phase oxidation of aromatic hydrocarbons such as benzene, the xylenes, naphthalene, toluene, durene or β-picoline. In this way, it is possible to obtain, for example, benzoic acid, maleic anhydride, phthalic anhydride, isophthalic acid, terephthalic acid, pyromellitic anhydride or niacin.

The process for preparing benzoic acid, maleic anhydride, phthalic anhydride, isophthalic acid, terephthalic acid, pyromellitic anhydride or niacin is generally known to those skilled in the art.

In the case of phthalic anhydride catalysts, it is shown in the examples that the catalyst according to the invention has the following advantages over the comparative catalyst (see Table 1):
a better phthalic anhydride (PA) yield and
a longer life (able to be estimated from the position of the hot spot).

### EXAMPLES

### A. Production of the Catalyst

A.1 First Catalyst Zone:

Suspension 1:

150 kg of steatite in the form of rings having dimensions of 8 mm×6 mm×5 mm (external diameter×height×internal diameter) were heated in a fluidized-bed apparatus and sprayed with 24 kg of a suspension comprising 155.948 kg of anatase having a BET surface area of 21 m²/g, 13.193 kg of vanadium pentoxide, 35.088 kg of oxalic acid, 5.715 kg of antimony trioxide, 0.933 kg of ammonium hydrogenphosphate, 0.991 g of cesium sulfate, 240.160 kg of water and 49.903 kg of formamide together with 37.5 kg of an organic binder comprising a copolymer of acrylic acid/maleic acid (weight ratio=75:25) in the form of a 48% strength by weight aqueous dispersion.

Suspension 2:

150 kg of the coated catalyst obtained were heated in a fluidized-bed apparatus and sprayed with 24 kg of a suspension comprising 168.35 kg of anatase having a BET surface area of 21 m²/g, 7.043 kg of vanadium pentoxide, 19.080 kg of oxalic acid, 0.990 g of cesium sulfate, 238.920 kg of water and 66.386 kg of formamide together with 37.5 kg of an organic binder comprising a copolymer of acrylic acid/maleic acid (weight ratio=75:25) in the form of a 48% strength by weight aqueous dispersion.

The weight of the layer applied was 9.3% of the total weight of the finished catalyst (after heat treatment at 450° C. for one hour). The catalytically active composition applied in this way, i.e. the catalyst shells, comprised on average 0.08% by weight of phosphorus (calculated as P), 5.75% by weight of vanadium (calculated as V₂O ₅), 1.6% by weight of antimony (calculated as Sb₂O₃), 0.4% by weight of cesium (calculated as Cs) and 92.17% by weight of titanium dioxide.

A.2 Second Catalyst Zone:

150 kg of steatite in the form of rings having dimensions of 8 mm×6 mm×5 mm (external diameter×height×internal diameter) were heated in a fluidized-bed apparatus and sprayed with 57 kg of a suspension comprising 140.02 kg of anatase having a BET surface area of 21 m²/g, 11.776 kg of vanadium pentoxide, 31.505 kg of oxalic acid, 5.153 kg of antimony trioxide, 0.868 kg of ammonium hydrogenphosphate, 0.238 g of cesium sulfate, 215.637 kg of water and 44.808 kg of formamide together with 33.75 kg of an organic binder comprising a copolymer of acrylic acid/maleic acid (weight ratio=75:25) until the weight of the layer applied was 10.5% of the total weight of the finished catalyst (after heat treatment at 450° C. for one hour). The catalytically active composition applied in this way, i.e. the catalyst shell, comprised on average 0.15% by weight of phosphorus (calculated as P), 7.5% by weight of vanadium (calculated as V₂O₅), 3.2% by weight of antimony (calculated as Sb₂O₃), 0.1% by weight of cesium (calculated as Cs) and 89.05% by weight of titanium dioxide.

B. Oxidation of o-xylene to PA-13 Model Tube Test of the Catalyst

B.1 Filling of the Model Tube

1.30 m of the catalyst A.2 and 1.70 m of the catalyst A.1 were in each case introduced from the bottom upward into a 3.5 m long iron tube having an internal diameter of 25 mm. The iron tube was surrounded by a salt melt to regulate the temperature, and a 4 mm external diameter thermocouple sheath (maximum length 2.0 m from the top) with an installed withdrawable thermocouple served for measurement of the catalyst temperature.

B.2 Preactivation of the Catalysts

The catalyst was installed and preactivated as follows: heating from room temperature to 100° C. under an air stream of 0.5 standard m³/h, then from 100° C. to 270° C. under and air stream of 3.0 standard m³/h, then from 270° C. to 390° C. under and air stream of 0.1 standard m³/h and holding at 390° C. for 24 hours. After this preactivation, the temperature was reduced to 370° C.

B.3 Start-up of the Catalysts

In test 1 (according to the invention), 3.0 standard m³/h of air having loadings of 99.2% strength by weight o-xylene of 30-40 g/standard m³ were passed through the tube from the top downward for 20 hours to start-up the catalysts. After 20 hours, the amount of air was increased to 4.0 at the same loading. The loading was increased to 80 g/standard m³ over a period of 20 days.

In test 2 (comparative example), 4.0 standard m³/h of air having loadings of 99.2% strength by weight o-xylene of 30-40 g/standard m³ were passed through the tube from the top downward for 20 hours to start-up the catalysts. The loading was increased to 80 g/standard m³ over a period of 20 days.

B.4 Oxidation of o-xylene to Phthalic Anhydride

4.0 standard m³/h of air having loadings of 99.2% strength by weight o-xylene of from 30 to 80 g/standard m³ were passed through the tube from the top downward. At 80 g of o-xylene/standard m³, the results summarized in Table 1 were obtained (“PA yield” is the amount of phthalic anhydride obtained in percent by weight, based on 100% pure o-xylene).

TABLE 1
Model rube results for the oxidation of o-xylene to phthalic anhydride,
started up using two different amounts of air (3.0 and 4.0 standard m³/h).
The phthalide content is below 0.15% by weight.

| | Test 1 | |
|---|---|---|
| Model tube results | according to | Test 2 |
| Catalyst A.1 | the invention | comparative example |
| Start-up amount of air | 3.0 | 4.0 |
| [standard m³/h] | | |
| Amount of air during | 4.0 | 4.0 |
| operation [standard m³/h] | | |
| Start-up temperature [° C.] | 370 | 370 |
| Start-up loading [g/standard | 30-40 | 30-40 |
| m³] | | |
| loading during operation | 80 | 80 |
| [g/standard m³] | | |
| Running time [days] | 27 | 27 |
| Salt bath temperature [° C.] | 355 | 356 |
| Position of hot spot in the | 13 | 17 |
| catalyst bed [%] on the 4th | | |
| day | | |
| Hot spot temperature [° C.] on | 440 | 440 |
| the 4th day | | |
| Position of hot spot in the | 27 | 30 |
| catalyst bed [%] under | | |
| operating conditions | | |
| PA yield [m/m-%] | 114.2 | 113.8 |

1-8. (canceled)

9. A method comprising: providing an oxidation catalyst bed; and starting up the oxidation catalyst at a temperature of 360° C. to 400° C. with an amount of air of 1.0 to 3.5 standard m³/h and a hydrocarbon loading of 20 to 65 g/standard m³, such that a hot spot having a temperature of 390° C. to <450° C. is formed in the first 7-20% of the catalyst bed.

10. The method according to claim 9, wherein the amount of air is 2.5 to 3.5 standard m³/h.

11. The method according to claim 9, wherein the amount of air is 3.0 to 3.3 standard m³/h.

12. The method according to claim 9, wherein the hydrocarbon loading is 30 to 55 g/standard m³.

13. The method according to claim 10, wherein the hydrocarbon loading is 30 to 55 g/standard m³.

14. The method according to claim 11, wherein the hydrocarbon loading is 30 to 55 g/standard m³.

15. The method according to claim 9, wherein the hydrocarbon loading is from 30 to 45 g/standard m³.

16. The method according to claim 10, wherein the hydrocarbon loading is from 30 to 45 g/standard m³.

17. The method according to claim 11, wherein the hydrocarbon loading is from 30 to 45 g/standard m³.

18. The method according to claim 9, wherein a hot spot having a temperature of 420 to <450° C. is formed in the first 10-20% of the catalyst bed after 24 hours.

19. An oxidation catalyst bed prepared by the method according to claim 9.

20. An oxidation catalyst bed prepared by the method according to claim 13.

21. An oxidation catalyst bed prepared by the method according to claim 17.

22. A method comprising: providing an oxidation catalyst bed according to claim 19; and oxidizing a starting material in the presence of the catalyst bed to form one or more products selected from the group consisting of benzoic acid, maleic anhydride, phthalic anhydride, isophthalic acid, terephthalic acid, pyromellitic anhydride, and niacin.

23. A method comprising: providing an oxidation catalyst bed according to claim 20; and oxidizing a starting material in the presence of the catalyst bed to form one or more products selected from the group consisting of benzoic acid, maleic anhydride, phthalic anhydride, isophthalic acid, terephthalic acid, pyromellitic anhydride, and niacin.

24. A method comprising: providing an oxidation catalyst bed according to claim 21; and oxidizing a starting material in the presence of the catalyst bed to form one or more products selected from the group consisting of benzoic acid, maleic anhydride, phthalic anhydride, isophthalic acid, terephthalic acid, pyromellitic anhydride, and niacin.
